(19) United States
(12) Patent Application Publication
(10) Pub. No.: US 2025/0375251 A1
(43) Pub. Date: Dec. 11, 2025

(54) NEW COMPONENTS DISPOSABLE AND MULTI-USE RE-STERILIZABLE FOR THE ROBOTIC SYSTEM FOR ENDOVASCULAR SURGERY

(71) Applicant: Guido DANIELI, Rende (IT)

(72) Inventors: Guido DANIELI, Rende (IT); Salvatore DE ROSA, Calvizzano (IT); Giovanni CARBONE, Sellia Marina (IT); Pasquale Francesco GRECO, Carolei (IT); Gabriele LAROCCA, Polistena (IT)

(73) Assignee: Guido DANIELI, Rende (IT)

(21) Appl. No.: 18/878,469

(22) PCT Filed: Jun. 27, 2023
(86) PCT No.: PCT/IB2023/056635

§ 371 Date: Dec. 23, 2024

(30) Foreign Application Priority Data
Jun. 28, 2022 (IT) 102022000013684
Apr. 18, 2023 (IT) 102023000007506
Apr. 18, 2023 (IT) 102023000007512
Jun. 26, 2023 (IT) 102023000013104

(51) Int. Cl. A61B 34/30

(52) U.S. Cl. CPC A61B 34/30; A61B 2034/301; A61B 2034/302

(57) ABSTRACT

The present patent application deals with the new components disposable and multi-use re-sterilizable for the robotic system for endovascular surgery ROSES that must be used for the new Robotic Actuators with passage hole of 36 mm in diameter, suitable for angioplasty and for catheters and guide wires of any dimension and purpose, included a new rotating hemostatic valve, a new double curvature animated catheter and the mechanisms controlling the configuration of the two types of animated catheters with one and two curvatures controllable from console.

### STATE OF THE ART

There are at least three robotic systems on the market, two of which are mainly dedicated to angioplasty and one to the whole endovascular field, but without extension to angioplasty. The first, CorPath from the American Corindus (Massachusetts), is characterized by large disposable components which, to measure the opposing force from the patient's body to the advancement of guides and catheters, even contains the sensor as well as the motors. The second, Robocath, French, is a kind of copy of CorPath, hence sharing its strengths and weaknesses. The third, Magellan of Hansen Medical, of Silicon Valley (California), has a rather complex system, and for the moment it is not very widespread in Europe.

This Patent/PCT application concerns new disposable and re-sterilizable accessories for the ROSES system (RObotic System for Endovascular Surgery), and collects what has already been presented in the Italian patent applications 102022000013684 of Jun. 28, 2022, 102023000007506 of Apr. 18, 2023, 102023000007512 of the same date and 102023000013104 of Jun. 26, 2023. Our robotic system has Robot Actuators of non-negligible dimensions, but in the direction perpendicular to the advancement of the catheters, while in the direction of work, between disposable and Actuator, the dimensions remain limited. These disposables are made by two separate elements, upper and lower, and, after removing them, a sterile passage of 36 mm in diameter remains, through which almost any accessory used in endovascular surgery can pass, to which a special hemostatic valve is now added in some cases, especially useful for angioplasty. The disposables do not contain motors, but receive motion from the Robot Actuator which allows three control parameters, the rotation of the whole disposable, plus two others which, for example in the case of angioplasty, control the advancement and retraction of guide and catheter, in a completely independent way. Furthermore, the forces opposed by the human body to the advancement of guides and catheters are measured thanks to a proprietary system of the ROSES system (first Italian patent already granted, and soon the European patent will be granted too), which is fixed to the robotic system and provides the measures even if the doctor is not interested, which obviously does not happen, since it is a system that increases the safety of the system.

It is also possible to produce a double Robot Actuator (PCT/IT2021/000042) which, on the two faces, can provide the control of six independent parameters, two main rotations and two other rotations on each side, obtained by means of two protruding shafts on both sides by two main wheels and relative rotating frames, shafts that can end with two bevel gears or with faceted shafts in order to transmit the necessary torques for example to friction wheels, almost always through conical couplings. In reality, the first version of this double actuator will be limited to 5 degrees of control, including two rotations, even if it has been studied and will be presented a system with 6 degrees of control, that will be useful for a new animated catheter, which presents two animated curvatures, that can be in the same or opposite direction, as commanded by the operator. Finally, an actuator with only two degrees of control has also been developed for which a single-use particular has been studied that is introduced into the passage hole, presented in the Italian patent application 102021000016946 of Jun. 29/21, which we will use as Proximal actuator with relative internal disposable on the measuring platform containing the double actuator in the distal position. Everything will also be illustrated in the figure in order to understand the use, and therefore the need for some particularities of the disposables that will instead be claimed, even if there will be no claims relating to the platform or to the new Actuators since they are simplified versions. than what has already been presented as a patent application.

The new disposable, derive from the disposable for ROSES already presented in the Italian application 102021000022490 of Aug. 30, 2021, and in the subsequent application of PCT/IT2021/000042 of the same date for big size catheters, and whose images were actually presented in an article, IJBTT-V1113P601, of 10 Jul. 2021 concerning the development of a robot to separate doctor and patient during intubation, however without going in depth into the details of how it was actually made, what was then done in the PCT. From this first derives also a new disposable for intermediate catheters, in which the typical adjustment screw is replaced by a simple spring using a single conical wheel, while another four new disposables have been developed, which take up the methods of fastening to the Robot Actuator, but they are still different and innovative.

The first of these new disposables is dedicated to the standard 0.035″ guides wires, and maintains the same dimensions of the lower element of the previous disposable for even of large dimensions catheters, but takes its motion from the right bevel gear, and changes both the lower friction wheel, and the upper one, which also becomes of fixed geometry. The other three are dedicated one to angioplasty and the other two to guide wires with movable core of the two types currently available, one with a core fixed at the tip, which allows to cancel the initial curve typical of the guides, the other with a core free to move with the rigid end that also allows you to straighten the initial curve. In all these three cases, however, while the method of attachment to the robotic actuator remains unchanged, the body of the disposable is lengthened since it houses wheels smaller in size but in higher number than that for catheters. Furthermore, the disposable for angioplasty has a second tube with an internal diameter of less than 6 mm, inside the tubular part so as to limit the lateral displacements of the guides which, as is known, are very thin. This channel, as will be shown, is in reality part of the new hemostasis valve.

It should be noted that thanks to the particular dimensions of the passage hole through the Robot Actuator using the same type of attachment to the RA, it will be possible to develop new single or multi-use elements to control, for example, the movement of catheters for ablation or of any other type, allowing in all cases to keep the doctor at the right distance from the radiant sources. Finally, the rotary hemostatic valve for angioplasty disposables and usable also for disposables with mobile cores, and the new command devices for animated catheters with one and two degrees of curvature control, of which the second is an absolute novelty, are also presented. especially useful for endovascular in large vessels.

### DESCRIPTION OF THE PREFERRED EMBODIMENT

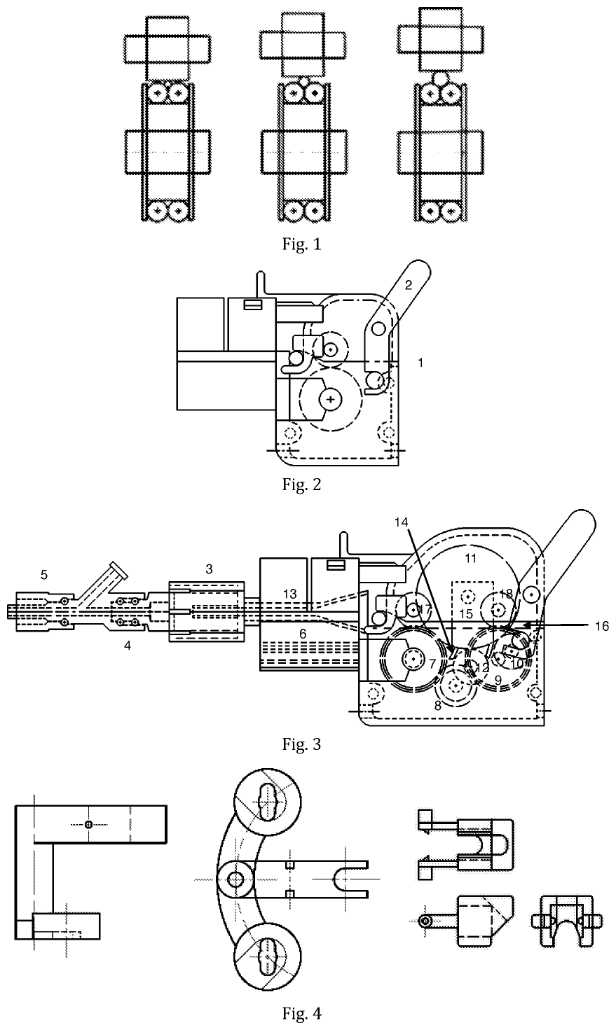

Therefore, starting from the disposable for catheters of usual dimensions, from 4 to 9 French, we illustrate the new version which differs from the previous one because the thrust screw is replaced by a spring, leaving two 4 mm of section silicone O rings as cover for the lower friction wheel, juxtaposed, while the counter-wheel consists of a flat silicone ring, which precisely allows, with small excursions of the spring, to accommodate catheters of various sizes. The detail of the two friction wheels for catheters is illustrated in FIG. 1. Using this system for the lower friction wheels, the upper wheel should lift less than two millimeters when passing from a 4 French to a 9 French catheter. The same system was then also adopted for the internal disposables for the actuator robots with only two degrees of control, but the difference is limited to the upper element.

Moving on instead to the 0.035″ guides, the disposable differs from the one presented in the aforementioned patent application first of all because the friction wheels are both covered by a flat silicone ring, given that the dimensions are almost constant and the guides themselves are much less deformable of the catheters, while the bevel gear used is the one on the right. In addition, the top member extends from the outset downwards by 3 mm beyond the axis of rotation of the main RA gear train to allow for placement of the small friction wheels in the top member, while correspondingly lowering by the same amount. lower element, so the two components match perfectly (1). This modification will then be present in all subsequent disposables that are illustrated here.

Again, with reference to the disposable for catheters mentioned in the patent application No. 102021000022490 of Aug. 30, 2021, the external appearance of the upper element also changes a lot, since there is no longer the adjustment screw since the upper wheel is mounted in fixed position, while the usual closing hook (2) is present. This is shown in FIG. 2.

Before moving on to the new disposable for angioplasty, let us first point out the profound difference between the grasping methods of the 0.035″ guides, which are larger and more knurled as they are made from a cable wrapped in a very tight spiral, for which two wheels covered with silicone are enough to transmit torsion and advancement, and those of 0.014″, which are formed by a very thin metal cable, for which it is necessary to pass the cable through a complex path described in the following.

Then there is a second potentially big problem to solve, the fact that while initially catheter and guide are internal to each other, this is no longer true about 20 cm from the tip of the catheter. Hence during the operation these present different access points within the hemostatic valve. This causes, in case of rotation of the disposable, the wrapping of the guide, thinner, around the catheter, reducing the possibility of rotation of the same. Therefore, thinking with my co-inventors, we hypothesized the need to also rotate the hemostatic valve with respect to the disposable. However, this also involves the need to maintain both the position of the initial catheter, and that of the radio-opaque liquid tube. This entails the use of a special hemostatic valve, which will be mounted at the tip of the disposable, which it will easily pass through thanks to the new dimensions of the passing hole, while its end portion must in any case be blocked to prevent their rotation, using a support that was already present to keep the whole valve fixed, while now it will only fix the terminal part. With reference to FIG. 3, (3) is the valve container, fixed to the disposable, while the valve will open by pressing the internal tube towards the RA, free to move with respect to the main part of the disposable, guided by a kind of rail as will be explained in the following, being (4) the joint that allows the relative rotation between the two elements, while (5) is the part to be fixed to the RA. Last note on this topic: since in reality as will be illustrated later, the rotating hemostatic valve could be used, before the angioplasty procedure, also for a guide with a movable core, it was decided that it is better if this can be detached from the tube of passage of guides and catheters, but to ensure that the valve itself rotates together with the disposable, it was decided to make the final part of the disposable detachable using a cross graft that will be better illustrated later, limiting ourselves at the moment to indicate its presence with dashed lines (6). However, if the system is also used for the introduction of the first catheter, it is clear that the same must be able to rotate during introduction, but once introduced, the system must stop to keep the catheter in position. Therefore, during this phase, it is necessary that the support of the hemostatic valve rotates with the catheter, which must be attached to the rotating frame, and this support is shown in FIG. 4, seen from the side and from above, with the small cap that can be fixed to the support itself next to it to block the rotary hemostatic valve. Of course, once the first catheter is positioned, the catheter must no longer be moved, while the disposable for angioplasty must be able to rotate smoothly. And, in this case, it is necessary to use the double Actuator Robot, called RA5, with reference to the number of motors present inside the RA, because once the catheter has reached the right position, the second gear stops, while the other can allow the disposable all the appropriate rotations.

Let us now start examining the lower thrust element for angioplasty, noting that both the catheters and the 0.014″ guides are very thin. This element has a basic structure composed of three sub-elements, two lateral ones plus a central one, that separates the thrust side of the catheters (on the left) from that of the guides, sub-elements that are assembled to form a single element, containing a series of shafts and gears, two of which are bevel to connect to the catheter and guide wire friction wheels. We note that the side dedicated to the catheter contains, on the axis of the bevel gear, in addition to the friction wheel, also a spur gear with 26 teeth, module 0.75 (7), which meshes with an idle gear with 15 teeth (8) to transmit motion to a second 26 teeth gear (9) coupled to the second friction wheel for the catheter.

On the guide wire's side, on the other hand, in addition to the friction wheel fixed to the bevel gear, of the same size as those for the catheter, and therefore not expressly indicated since in this view the two wheels coincide, there are also two other friction wheels, one rear it has a diameter of 11 mm (10) to replace the previous wheel with a diameter of 19 mm connected by means of an idle wheel to the gear integral with the bevel gear for motion transmission. While in the previous disposable for angioplasty there were two friction wheels for the guide, which were on the upper thrust element, and touched the two friction wheels connected by the gears in internal points, now the two wheels are replaced by a single wheel with a diameter of 32 mm (11), including silicone, placed on the upper thrust element. This wheel touches the two friction wheels in exactly the same points where the two previous wheels touched, thanks to the smaller diameter of the new rear friction wheel. A third wheel of 11 mm (12) in diameter was then added in the middle to ensure that contact with the large wheel occurs in three points, increasing the continuity of contact, which is essential, even though there is no transmission of motion via gears.

To keep the wheels aligned, 4 mm diameter plastic shafts will be used, which rest on the 4 mm thick central support that divides the guide and catheter push lines, adding other internal supports, which will be different from the two sides. The central support then also supports the cursor on which is placed the removable central tube with an outer diameter 4, (13), in order to prevent the guides and catheters from winding up in a strange way. This tube will present initially a funnel shape. Furthermore, it is advisable that both at the entrance to the wheels and at the exit there are very narrow U-shaped slots, belonging to the lower element, so as not to leave the guide and catheter the possibility of moving laterally while a couple of further centering elements (14) fixed to an ad hoc support (15) surrounding the upper large wheel, they will replace the old bridge on the upper 32 mm diameter wheel for the guide wires.

It should also be noted that a channel (16) will be created on the top of the central element to accommodate a guide which should it have already reached a stenosis which is located near a bifurcation of the coronary arteries. In this case it is possible to position the guide and possibly the related catheter in this separation channel by removing them from the thrust path, to position a new guide in the disposable, and possibly a new catheter and introduce the guide into the bifurcation before inflating the stent. This allows you to place a second stent in the bifurcation, eliminating the second stenosis just generated, no longer making it necessary to use a second parallel Slave, as initially envisaged. As before, there will be two friction wheels 11 mm at the top for the catheter in correspondence with the lower friction wheels (17) and (18) on the driver side. Obviously the upper element hooks to the same lateral pins, and also presents the centering teeth from behind as in the previous model for large catheters. Note that the insertion of the large wheel in place of the two side wheels has allowed a considerable simplification and a much safer ability to turn the guide on itself. Note that the new nose, despite the larger dimensions in the transverse direction, has not added length, and how, even when open, the large wheel does not disturb the maneuvers.

| Coordinates of the axes of the | | | |
|---|---|---|---|
| various wheels | r | x | y |
| Upper Friction wheel guide wires: | 16 | 33.8 | 4.5 |
| Friction wheels driven by bevel gears: | 9.5 | 17.0 | −14.8 |
| Catheter upper friction wheel: | 5.5 | 17.0 | 0.3 |
| Second friction wheel guide wire: | 5.5 | 47.1 | −12.4 |
| Third friction wheel guide wire: | 5.5 | 34.6 | −17.0 |
| Second lower friction wheel catheter: | 9.5 | 43.0 | −14.8 |
| Second upper friction wheel catheter: | 5.5 | 43.0 | 0.3 |
| Idle spur gear module 0.75, 15 teeth: | | 30.0 | −23.0 |
| Upper Friction wheel guide wires: | 15 | 33.7 | 3.1 |
| Third friction wheel guide wire: | 5.5 | 34.6 | −17.4 |
| Upper Friction wheel guide wires: | 14 | 33.7 | 1.8 |
| Third friction wheel guide wire: | 5.5 | 34.6 | −17.7 |
| Upper Friction wheel guide wires: | 17 | 33.8 | 5.7 |
| Third friction wheel guide wire: | 5.5 | 34.5 | −16.8 |
| Upper Friction wheel guide wires: | 18 | 33.9 | 7.0 |
| Third friction wheel guide wire: | 5.5 | 34.5 | −16.5 |

We note that there are four friction wheels both on the catheter side and on the guide side, of which only those aligned to the axis of the bevel gears are coaxial, while all the others are in different positions, not aligned, and whose axes they do not interfere in the least. Summarizing the coordinates of the axes in view x, y are, according to the radius of the upper friction wheel for guides, provided in the previous table, in order to allow perhaps initially to produce three different disposables with upper wheel with a variable diameter from 28 to 36 mm, to choose the one that pushes best without deforming the guide. Note that in the table, while the coordinates of all axes are provided for the 32 mm friction wheel, changing the friction wheel only changes the coordinates of the center of the upper wheel and those of the lower central wheel, while the other data remain unchanged.

However, we note that, as illustrated in the last PCT presented on 30 August, PCT/IT2021/000042, a new double Slave has been developed, containing two independent gear trains aligned on the same axis and always with a passage of 36 mm in diameter, capable to control the usual disposables in the rear part, such as the one for angioplasty just presented, while the anterior gearing allows to rotate both the fixed part of the hemostatic valve and to control a future variable curvature catheter. In addition, a new Slave with two degrees of freedom was also developed, deriving from ROSINA (RObotic System for INtubAtion) 102021000016946 of Jul. 2, 2021, which contains the thrust element inside the tube itself. Finally, the patent application EP20737294-7 contains a system, now in the industrialization phase, which allows both the measurement of the forces opposed to the advancement in the body of guides and catheters, and to move the relative position between a proximal Slave, held still by being placed on a sled on an inclined rail but fixed to a force transducer and a movable distal.

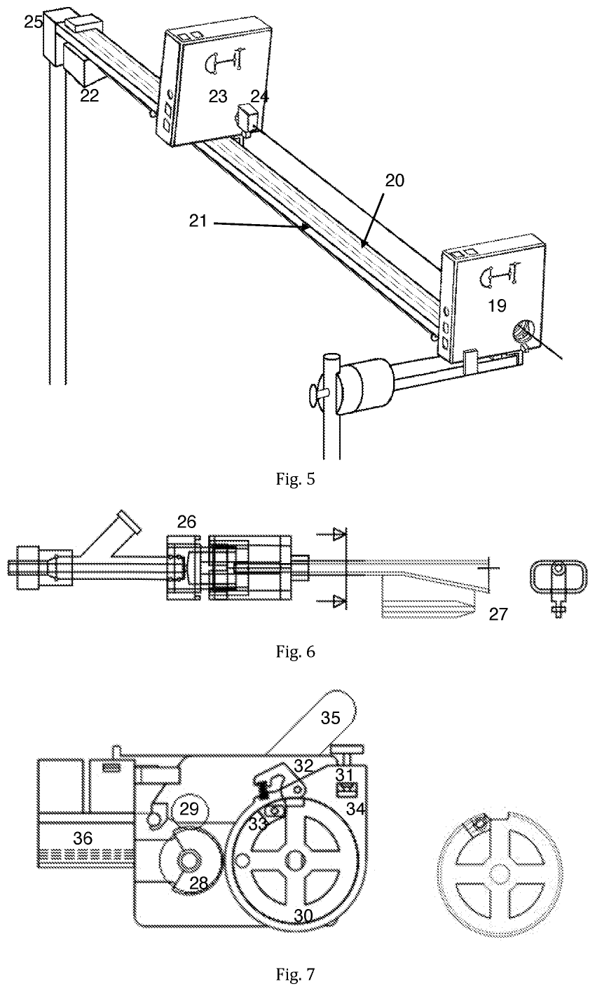

Now, putting these three devices together we get the system, illustrated in FIG. 5, so that by placing a preformed catheter, like the initial catheters used in angioplasty, connected to the final part of a rotating hemostatic valve, while from behind a disposable for guides with movable core is introduced, which naturally has the same central tube and relative hemostatic valve as the disposable for angioplasty, it is possible to bring the initial catheter into position also in this case by working at a distance. Then the guide and disposable for guide with movable core are extracted, leaving the tube connected to the hemostatic valve in position, the disposable for angioplasty is introduced without final tube and hemostasis valve, remained in the double Slave, and the new procedure is started, obviously without touching the position of the initial catheter which, having changed the program, it no longer has handling. In particular (19) it is the proximal Slave, placed on a slide free to move on a rail (20)) as long as the stainless steel base (21) while a motor placed under the bar (22), fixed to the proximal Slave from an invisible side bar, moves the distal double Slave (23) through a worm screw, free to rotate the part of the hemostatic valve (24) with respect to the fixed part, while a transducer (25) connected to the motor, detects the forces acting in the system.

However, we note, with regard to the hemostatic valve, which was sincerely inspired by competitor valves, that these valves also have the possibility of varying the force with which the valve opposes the passage of guides and catheters, thus creating more or less internal friction to the system, which therefore does not interest us from the point of measuring the forces. Now this variation is obtained in commercial valves that have this characteristic, by rotating the final body of the valve with respect to the octagonal element, which advances the valve with respect to the internal tube thanks to a very fine thread, which is what, passing through the silicone valve element, opens the passage. Reflecting on this system, which we have also adopted, we understood that in reality our valve could have rotated on the thread instead of on the joint that we prepared. To remedy this possible problem, we added an octagonal element (26) of FIG. 6 that can slide with respect to the valve side of the joint that joins the valve part to the radiopaque liquid inlet and catheter outlet, and can be inserted in the external octagonal part, so that this allows the valve closing force to be adjusted, but once regulated, it blocks further possible rotations, leaving only the waterproof joint set up for relative rotations to rotate. Note also the peculiar spearhead shape (27) of the inlet part of the valve system in the disposable, in order to facilitate its exchange with the disposable for guides with mobile. Furthermore, it should also be noted that this system also allows, by pressing the funnel part, to open the hemostatic valve, compressing the internal spring and making the central tube penetrate into the silicone part, widening the passage hole.

Let's now pass to describe the two disposables for 0,035″ guide wires, but with movable core, starting the description with the one that has the external lateral wheel on which the core of the wire is wound to make the rigid part move, (FIG. 7). We note that in this case, while maintaining the elongated dimensions of the disposable body, the second and third friction wheel of the right body have been eliminated by leaving as obvious the two shafts aligned ending with the bevel gears (28) with the one on the left that carries s 15 teeth spur gear the transit motion to the second and third wheels of 26 and 15 teeth for the motion of the movable core, placing the friction counter-wheel on top of the upper element (29) on the right side, for the thrust of the guide, as in the previous case. On the left-hand portion, the last toothed wheel transmits the motion on the axis, which comes out of the lateral support, to an external wheel (30) of adequate size so as not to disturb the bevel gear that transmits the motion. A small clamp (31) of the side element of this disposable was then added to fix the body of the guide wire aligned with the entrance of the wheel, while the internal thread is introduced into the large wheel by pressing the tilting element with spring (32) which comes out of the support outside the core winding wheel, where it is fixed by turning a screw placed on the same wheel (33). In doing so, by turning the outer wheel, the core of the guide is wrapped around a channel that surrounds the entire wheel closed externally by a fixed cover, also integral with the lateral shell (34), which forces the wire to remain from its seat when the movement is reversed, exiting only at the point where the wire itself enters. It should also be noted that the external fastening hook of the upper element to the lower one is in this case present only on one side (35). Finally, note the presence of the dashed lines (36) that represent the rail to insert the central tube which holds the usual rotating hemostatic valve system, as in the case of the disposable previously illustrated.

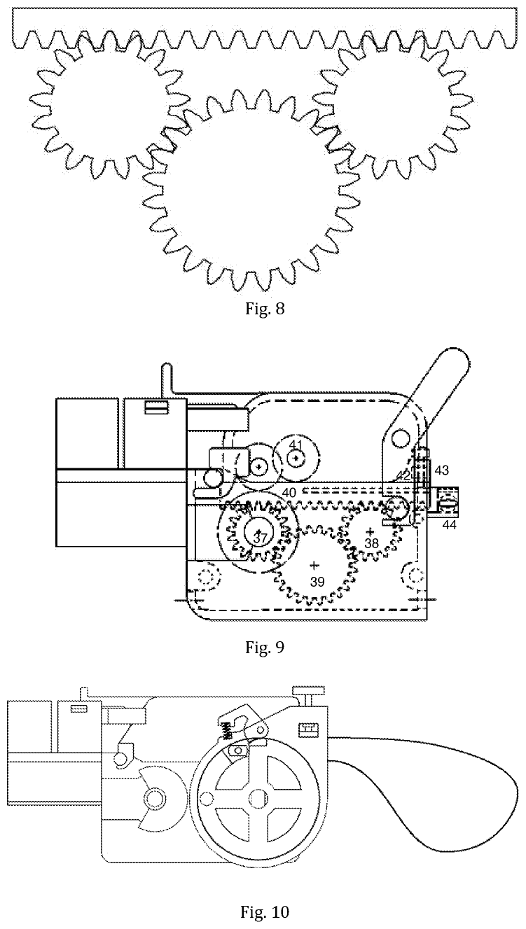

Moving on to disposable with fixed core at the tip, also in this case the configuration previously used for disposables for angioplasty of the three toothed wheels, one of which is idle, has been restored, but only for the side where the rack is present, so that there are two toothed wheels turning in the same direction with a number of teeth equal to that previously used as a central gear which mesh with an intermediate gear having the same number of teeth as that of the previously used gears coupled to the friction wheels, thus maintaining the same previous wheelbase. The reason for this inversion of the gears is to obtain an increase in the force transmitted to the wire, which while in the previous case the force required for handling was very low, for the core fixed at the tip the necessary force is much greater. The problem of the positioning of the wheels was solved using Newton Raphson whose solution is represented in FIG. 8, which shows the profiles of the three gears and relative rack, obtained by calculating the profiles of the involute teeth and the relative meshing and plotting the result with Excel.

The distance between the centers of the upper wheels was used as a free parameter, keeping the rack horizontal engaged with the first wheel. In conclusion, the following coordinates were found for the axes of the three gears, identical to the positions of the axes in the previous cases. Better, these are the axes positions found with the just described system, which was close to the values initially assumed for the axes, which were then assumed identical for the three disposables.

| | | | |
|---|---|---|---|
| x | 17 | 29.9 | 42.8 |
| y | −14.75 | −23.1 | −14.75 |

With reference to FIG. 9 which shows the disposable in side view, the two 17-tooth gear shafts (37) and (38) are held in position by a third bigger diameter gear (39) and at the same time ensuring that the rack (40) does not come out laterally (the first gear is obviously coaxial to the bevel gear, from which it takes the motion), two lateral thin surfaces have been added fixed on the shafts of the second and third gear and also fixed together to create a rectangular passage for the rack that will start from the first gear toward the patient. An idle wheel will be placed on the rack (41), belonging to the upper element, which prevents the rack from rising, while in the rack itself a 1 mm hole will be drilled along the axis of motion, in order to insert the guide, while at the beginning of the rack there will be a swelling (42) which will prevent the rack from passing the upper wheel, in which a screw will be inserted, passing through a metallic threaded insert (43) which will tighten the head of the guide locking it. Then there will be a small clamp (44) externally which tightens the body of the wire itself. While the upper cover has both a fixed friction wheel (45) above the push wheel connected to the lateral bevel wheel, and the aforementioned friction wheel above the rack (40) which allows both to keep it in position and to limit its stroke, since the cover is always equipped with all the elements both in the front and in the rear part able to lock it in position, as described in the aforementioned Italian patent application.

Finally, FIG. 8 shows how these two last disposables work, which simultaneously allow the guides to be advanced and the internal core to be activated, simply by returning the tail of the guides to the disposable and fixing it to it, as is foreseen in the previous models developed for the RA with the small hole.

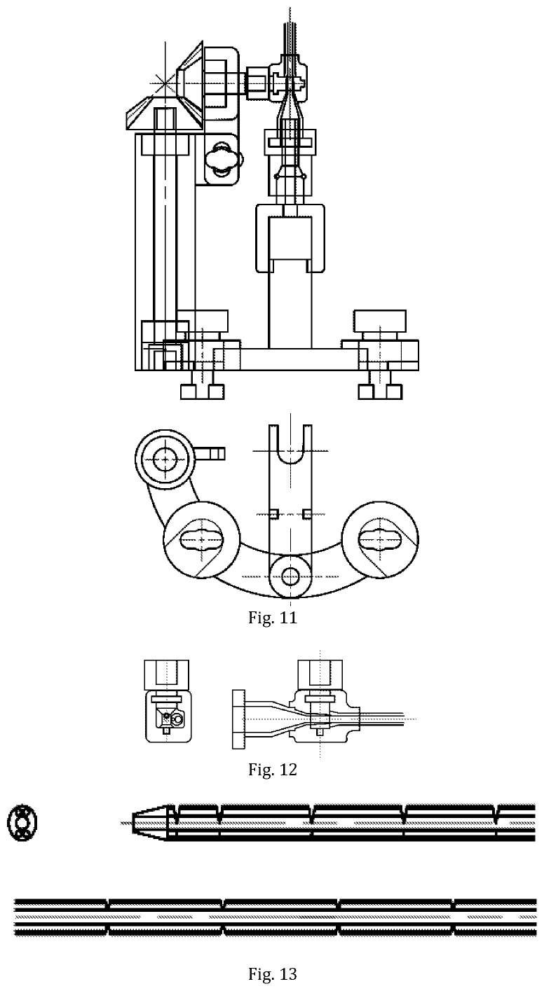

Finally, let's see how the use of the double Actuator Robot, RA5 or RA6, also allows you to control the animated catheters, both with variable curvature of the tip and with double curvature. Starting from those with curvature of the tip, FIG. 11 shows the mechanism which is mounted at the outlet of the second frame, which derives from the support of the hemostatic valve of FIG. 4, extended on one side to take the necessary motion from a faceted shaft which comes out of the frame.

From this starts a small column which takes the motion from the faceted shaft and brings it to the height of the connector of the cored catheter, to which it transmits the motion through a pair of conical wheels, a connector which contains a small drum on which the wire which moves the tip. Obviously, given that the mini drum must be placed laterally to the catheter, which is instead held in the center, the column, the axis that transmits motion to the column, is moved back from the normal to the central support by a couple of millimeters. FIG. 12 instead shows the connector into which the bevel wheel that transmits the motion is inserted.

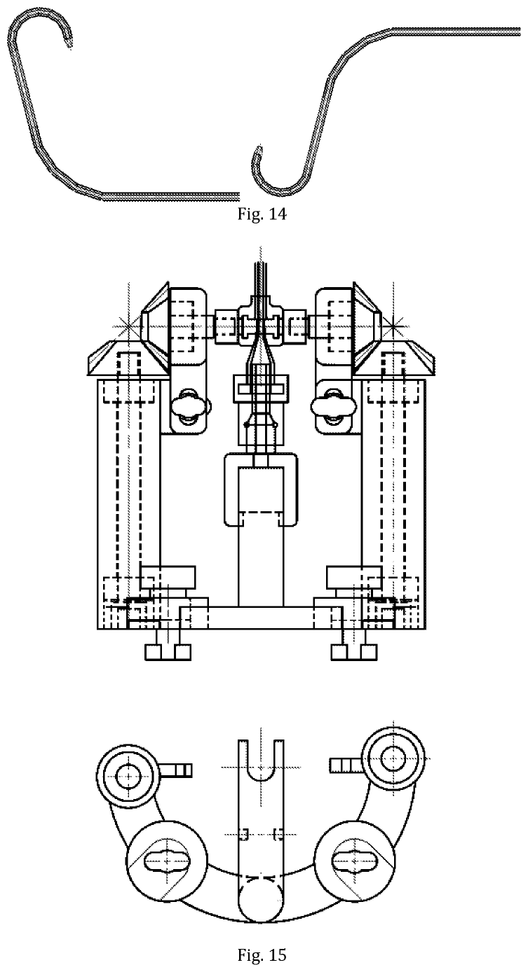

FIG. 13, on the other hand, shows the section and preparation methods of the parts of the double-curved catheter, which must contain, in addition to the central channel, four small lumens, because, while the catheter with the curved tip needed only one cable, being able to straighten the same with the mobile core of the guide, in this case it is better to provide cables that can act in both directions, while leaving a certain slack between the actuator cables on both sides. As can be seen, the methods of preparing the cuts are different, between the area of the toe, where curvature is foreseen only in one direction, and that of the body, where the cuts are symmetrical. Of course, the curvature obtainable depends on the distance between the cuts. FIG. 14 then shows two of the infinite possible configurations obtainable from these catheters.

Finally, FIG. 15 shows the mechanism that uses two differentiated and opposite outputs for the command of the movement of this catheter, in relation to which we also note that to move it we will need a joystick with four linear degrees of control, rotation, advancement and the two curvatures.

1. New Disposable and multi-use re-sterilizable components for the Robotic Actuator of the endovascular surgery robotic system for guides and catheters for various applications ranging from endovascular surgery to angioplasty, characterized by:
the presence of one of two bevel gears that take power from two bevel gears that exit from the main gear of the gear train or alternatively in the case of multi-use accessories that can be sterilized, by two shafts with a hollow initial part to take motion from two shafts that end with a suitable facet that protrude from a frame connected to the mother wheel of a second internal gear train,
the securing system to the Robotic Actuator is made of teeth that fit into special slots inside the disposable plate-tube, plus a locking key, such as those described in a previously cited patent application or even only with locking keys, in the case of connection to the rotating frame of a second train gear,
the possible presence of a special rotating hemostatic valve (only for disposables),
the fact that the disposables are composed of two elements, of which the first, the lower one, is connected to the bevel gear which protrude from the Robotic Actuator and which contain at least one friction wheel connected to a bevel gear, and the second, upper one, which contains the counterfriction wheels and anything else that is useful for the particular application,
being diversified according to the type and size of the medical device to be inserted, as in certain cases the presence of a rotating hemostasis valve, and certain others of active catheters, able to assume on command variable configurations;

2. New disposable components for the Robotic Actuator of the ROSES robotic system for endovascular surgery as per claim 1, characterized by the fact that:
the securing system to the Robotic Actuator of the lower components consists of two teeth placed on the upper fins of the centering tube of the same, which are placed in place by compressing two external bars connected to the upper fins, making the teeth enter two cavities for this purpose provided in the plate tube,
the securing system to the Robotic Actuator of the upper components has a central protuberance which is inserted into a special hole always present in the tube, by two arms which are inserted on two lateral pins which come out of the lower element, by two teeth which are inserted inside the lower element and finally by a hook that locks the two lower and upper elements together,
the key then locks the lower element together with the disposable tube plate by fixing them to the mother wheel from which the two bevel gears for feed control emerge:

3. Disposable components for the Robotic Actuator of the robotic system for endovascular surgery as per claim 2, in which the lower disposable component dedicated to pushing catheters with a diameter between 4 and 9 French has a friction wheel made up of two 4 mm section 0 rings, while the upper one, which instead has a silicone ring as a cover 8 mm wide, it is equipped with a spring that allows a small excursion of the tilting frame with increasing force, while for larger catheters, the spring is replaced by an adjustment screw;

4. Disposable components for the Robotic Actuator of the robotic system for endovascular surgery as per claim 1, wherein the disposable component dedicated to pushing standard 0.035″ guides in diameter, which is equipped with a bevel gear on the right side which transfers motion to a friction wheel covered by a flat silicone ring belonging to the lower element, while on the upper element there is a second small wheel covered with silicone that fits inside the two side wings of the lower friction wheel, locking the guide in position, this upper wheel being placed in a fixed position;

5. Disposable Component for the Robotic Actuator of the robotic system for endovascular surgery, as per the claim 1, wherein the disposable component designed for pushing guides and catheters for angioplasty, which is equipped with power take-offs and relative bevel gears on both sides, the right side being reserved for handling 0.014″ diameter guides and the left side for the characteristic small-sized catheters for angioplasty or similar applications, where on the right side on the driver's side there are three friction wheels of which only the first, larger one is motorized, while the other two, smaller ones and, are idle, but positioned so as to come into contact with a single wheel with a much larger diameter positioned on the upper component so as to force the guide, also thanks to a series of special centering elements present on both the lower and upper component, to assume a curvilinear configuration with three contact points which facilitates the transmission of torque to the guide to allow rotation, as well as advancement and retraction, while on the left side, dedicated to the catheters, there are two wheels covered with silicone rings that rotate together thanks to a lateral chain of spur gears, while on the upper element there are two friction wheels placed immediately above the lower ones so as to imprison the catheter between the silicone wheels, since there is also a channel between the sides of the guide and the catheter so to temporarily place the guide and catheter in case a bifurcation is found close to the stenosis, which could be closed by enlarging the stenosis with a stent, thus being able to position a new guide in the second branch of the bifurcation before inflating the balloon of the stent, thus being able to then position a second stent at the second branch, as there is also a central tube, easily removable if necessary, with a reduced diameter to guide catheters and guides up to introduction into the hemostatic valve, which is added at the tip, divided into three essential components, the valve itself, which rotates with the rest of the disposable, the body which must remain in a fixed position both for the catheter and for the connection to the inlet tubes of the radio-opaque liquid, and the joint which allows relative rotation between the two elements;

6. Disposable Component for the Robotic Actuator of the robotic system for endovascular surgery, as per the claim 1, wherein the disposable component designed for pushing a guide from 0.035″ with mobile core free to move inside the guide itself, in which, while on the right side there are the usual friction wheel connected to the bevel gear, with the friction counter wheel above it, on the left side there are no friction wheels, but only three toothed wheels identical to those present in the disposable for angioplasty, the last of which is connected to an external wheel on which the core inside the guide must be wound, covered by a circular structure which embraces the wheel itself, preventing it from coming out, while a clamp blocks the external body of the guide being the tail of the guide directed into a cavity inside the wheel and fixed there by moving a tilting clamp with spring in a special groove in the wheel itself where it is fixed by turning a screw placed on the
same wheel, finally being possible the presence of the removable tube containing the rotating hemostatic valve in case of use in conjunction with the disposable for angioplasty;

7. Disposable Component for the Robotic Actuator of the robotic system for endovascular surgery, as per claim 3, wherein the disposable component designed for pushing a guide from 0.035″ with mobile core fixed at the tip so that, by pulling the terminal on the operator's side of the guide itself, the initial curvature of the guide itself straightens, characterized by the presence of a rack, placed above two toothed wheels identical and whose position has been carefully studied which, through a third wheel, in this case larger than those which mesh with the rack to increase thrust, allows the movement of the rack itself which is connected to the mobile head of the guide characterized by a swelling, inserting it in a coaxial hole in the rack while a screw passing through a metal insert inserted in the distal end of the rack blocks the head, and an external clamp blocks the body of the guide, since the toothed wheels and the rack are surrounded on the sides by two thin walls, fixed to the pins of the two toothed wheels not fixed to the bevel wheel so as to prevent the escape from the underlying toothed wheels, being then the two lateral walls connected both from above and below beyond the proximal gear wheel to keep the rack straight even if pushed beyond the distal wheel, while an idle wheel placed on the rack prevents it from rising but only allows it to slide, being obviously the friction counter wheel is also present on the right side of the upper element, as in all the other thrust elements for 0.035″ diameter guides;

8. New disposable components for the Robotic Actuator of the robotic system for endovascular surgery, as per claim 1, in which the rotary valve is characterized by the fact that it differs from what is present on the market not only due to the presence of the waterproof joint which allows rotation between the actual silicone valve, contained within the octagonal element also present in the valves on the market, and the contrast liquid inlet connectors and the outlet on the catheter, but also due to the presence of a further octagonal element which allows to adjust the force with which the valves tighten on guides and catheters by rotating the outlet body on a thread with respect to the valve itself, but to block this rotation once the adjustment has been obtained, so that then the relative rotation between the parts of the valve can only take place on the joint arranged for this;

9. New disposable components for the Robotic Actuator of the robotic system for endovascular surgery, as per claim 1, consisting in an animated catheter characterized by the possibility of controlling from console the curvature both of the tip and of another portion of the body of the catheter, thanks to the presence of four small diameter lumens placed in couple on the two sides of the main lumen in which are present four cables, two fixed near the tip for its curvature control, and on the first available cut for controlling the curvature of the intermediate portion, being the curvature possible for the presence of mono-lateral wedge shape cuts near the tip and bilaterally on the catheter intermediate portion, being these cuts executed on the sides where the small lumens are present, so that while the tip can bend in one direction only, the curvature can be on both sides in the second section, being the proximity and number of these cuts element of control of the obtainable curvatures;

10. New re-sterilizable multipurpose accessories as per claim 9, which are attached to the rotating frame of the second train by means of suitable keys and take motion from a faceted shaft coming out of the frame itself, in the case of a 5-motor RA, to bring the winding control of the micro drum contained in the catheter connector with adjustable tip curvature, already mentioned in the technical literature, at the height of the same connector, placed at the outlet of the hemostatic valve, by means of a pair of conical wheels, the first connected to the faceted shaft and the second inserted at the exit of the connector drum, so as to allow winding and unwinding of the cable contained in the catheter, while in the case of a 6-motor RA there will be two columns surmounted by conical wheels which will act on the four cables contained by the double curvature catheter.
